(19) United States
(12) Patent Application Publication
(10) Pub. No.: US 2004/0220605 A1
(43) Pub. Date: Nov. 4, 2004

(54) Dilatation system

(75) Inventors: Carl-Sebastian Wagner, Bretten (DE); Manfred Weigel, Pforzheim (DE)

Correspondence Address: COHEN, PONTANI, LIEBERMAN & PAVANE, 551 FIFTH AVENUE, SUITE 1210, NEW YORK, NY, 10176, US

(73) Assignee: Richard Wolf GmbH

(21) Appl. No.: 10/811,555

(22) Filed: Mar. 29, 2004

(30) Foreign Application Priority Data
Mar. 28, 2003 (EP) 03 007 084.1

(51) Int. Cl. A61B 17/34

(52) U.S. Cl. 606/191

(57) ABSTRACT

The invention relates to a dilatation system consisting of a dilatation pin (20) with a diameter which increases proximally from the distal end, and with a tubular guide sleeve (2). The guide sleeve (2) has an inner diameter which corresponds to the diameter of the distal end (22), reduced in diameter, of the dilatation pin (20). Furthermore, the guide sleeve (2) in its longitudinal direction may be separated open over the entire length along at least one line (10). The invention further relates to a guide sleeve (2) for such a dilatation system.(FIG. 1)

### BACKGROUND OF THE INVENTION

The invention relates to a dilatation system as well as to a guide sleeve for such a dilatation system.

A dilatation of an opening to a body cavity is required if an existing or initially produced access with a smaller cross section is to be replaced by an access of a larger cross section. For example a veress cannula or trocar sleeve is firstly introduced, and later during the widening of the already created tissue passage a larger trocar or instrument sleeve is to be introduced in order to be able to carry out endoscopic operations. With this, a widening of the already created tissue passage is to be effected by way of dilatation or enlarging the cutting incision without the previously created tissue passage getting lost. If however the firstly introduced instrument, for example a trocar sleeve of a small diameter is removed in order subsequently for example to introduce a dilatation pin for widening the tissue passage, there exists the danger that the tissue passage closes again and may no longer be located after removal of the instrument.

For example from U.S. Pat. No. 5,431,676, U.S. Pat. No. 6,080,147 and U.S. Pat. No. 6,325,812 there are known trocar sleeves whose shank consists of a braiding which may be changed in diameter. This braiding may expand in order to create a larger, widened access. The disadvantages with these arrangements is the fact that wall thickness of such a trocar sleeve on introducing a veress cannula for example significantly increases the total diameter. Furthermore such a trocar sleeve of braiding is expensive in manufacture and on introducing a veress cannula for example demands an increased force effort.

### BRIEF SUMMARY OF THE INVENTION

It is therefore the object of the present invention to create an improved dilatation system which only slightly increases the total diameter on introducing an endoscopic instrument and which is inexpensive to manufacture and easy to clean.

This object is achieved by a dilatation system with the features specified in claim 1 as well as a guide sleeve for such a dilatation system, with the features specified in claim 11. Preferred embodiment forms result from the dependent claims.

The dilatation system according to the invention as an essential element comprises a dilatation pin as well as a tubular guide sleeve. With this, the dilatation pin has a diameter which increases in the proximal direction from the distal end so that the distal end is formed as a relatively thin guide lug. The diameter of the guide lug of the dilatation pin corresponds essentially to the thinner diameter of the tubular guide sleeve so that the dilatation pin with its narrow or reduced diameter end may be easily introduced into the guide sleeve. Additionally the dilatation pin at its distal end may be rounded off or chamfered in order to permit a simpler introduction into the guide sleeve. The guide sleeve despite the relatively thin wall thickness is designed intrinsically stable in a manner such that when it is inserted into an opening, for example an opening in the abdominal wall, it may maintain this opening without further elements having to be arranged in the inside of the guide sleeve. Furthermore the guide sleeve is designed such that it may be separated open over the whole length over along at least one line. This permits the removal of the guide sleeve after the dilation pin is introduced into the body opening. The dilatation system according to the invention permits the execution of a new dilatation method with which firstly the guide sleeve together with an endoscopic instrument such as for example a veress cannula or likewise is introduced into the tissue in order firstly to create an opening or an access with a small diameter. After the subsequent removal of the endoscopic instrument the introduced guide sleeve keeps the created opening open so that now the dilatation pin together with a trocar sleeve or likewise, which surround this, may be introduced into the guide sleeve. At the same time the dilatation pin due to its diameter increasing in the proximal direction widens the guide sleeve and the created opening. The guide sleeve is simultaneously separated open along at least one line over its whole length. If the access is completely dilated over its whole length and the guide sleeve is separated open over its whole length then this may be removed from the created, widened access, wherein this access may then be held open by the introduced dilatation pin or by the trocar sleeve surrounding this. The guide sleeve is designed as a throw-away part, by which means the cleaning expense and also the costs are significantly reduced.

The guide sleeve preferably has at least one break-off location extending in the longitudinal direction of the guide sleeve preferably over its whole length. Such a break-off location ensures that the guide sleeve tears open along a predetermined, defined line on widening by way of the dilatation pin.

The dilatation system further comprises a veress cannula with an outer diameter which corresponds to the inner diameter of the guide sleeve. At the same time the veress cannula may comprise a trocar sleeve or be arranged in the inside of a trocar sleeve whose outer diameter corresponds to the inner diameter of the guide sleeve. With such a veress cannula one may create the access to the body cavity preferably under optical control. At the same time the matching of the outer diameter of the guide sleeve and the outer diameter of the veress cannula permits these to be able to be introduced together into the tissue. For this the guide sleeve is firstly published onto the veress cannula and then together with this is pierced into the tissue. Subsequently the veress cannula may be retracted proximally from the guide sleeve, so that the guide sleeve alone remains in the created access or the created opening, and maintains this until introduction of the dilatation pin. In place of a veress cannula one may also use another corresponding suitable instrument for creating the access.

It is further preferred for two break-off locations preferably arranged diametrically opposite to be formed in the guide sleeve, which extend in the longitudinal direction of the guide sleeve. This design permits the guide sleeve to break up into two parts on widening by way of the trocar pin and subsequently to be removed more easily from the created access. One may however also provide more than two break-off locations distributed over the circumference, wherein the guide sleeve then on widening breaks up into three or more parts and may be accordingly withdrawn between the trocar sleeve and tissue.

The break-off locations are preferably formed by perforation or by weaker material of the guide sleeve at locations. The material of the guide sleeve at predefined locations may be formed weak in that it is designed thinner, i.e. the guide sleeve comprises a thinner wall thickness at the corresponding locations.

According to a special embodiment form the guide sleeve is formed from at least two sleeves which are separate from one another and arranged in one another and which in each case comprise a slot extending in the longitudinal direction of the guide sleeve preferably over its whole length or a correspondingly extending break-off location, wherein the slots or break-off locations in the two sleeves are arranged circumferentially displaced to one another. This arrangements has the effect that on widening by way of the dilatation pin both sleeves of the guide sleeve tear open or widen along the slot or the break-off location, by which means the arising slot widens. By way of the displaced arrangement of the break-off locations or slots over the circumference of the guide sleeve is achieved that the slot arising in the one sleeve in each case is covered by the walling of the other sleeve so that the guide sleeve on widening is always completely circumferentially closed. By way of this a walling of the guide sleeve is always arranged over the whole circumference of the dilatation pin, by which means a secure guiding of the dilatation pin is ensured and the guide sleeve despite this may tear open easily or is may be easily widened if the dilatation pin is introduced into the guide sleeve. If the guide sleeve consists of two sleeves arranged in one another, the slots or break-off locations formed in the two sleeves are preferably arranged displaced 180° to one another. In place of two sleeves one may also apply three or more sleeves. If the guide sleeve for example is constructed of three sleeves, their slots are preferably displaced each by 120° to one another on the circumference of the guide sleeve so that each slot is covered by the walling of another sleeve. The sleeves arranged in one another are preferably designed such that the respective inner sleeve with its outer circumference in each case bears on the next outer sleeve so that essentially no free space or place is present between the individual sleeves of the guide sleeve.

Preferably at least one holding element is formed at the proximal end of the guide sleeve. Such a holding element, e.g. a holding ring simplifies the handling of the guide sleeve. Thus on sliding in the dilatation pin the guide sleeve may be firmly held on the holding ring in order to prevent the guide sleeve being pushed into the body cavity by the dilatation pin. Furthermore the guide sleeve after widening by the dilatation pin may be easily gripped on the holding ring and be withdrawn from the created access. In place of a holding ring one may also provide another suitable grip element on the guide sleeve.

The guide sleeve is usefully manufactured of a preferably transparent plastic. The guide sleeve may be manufactured inexpensively of plastic as a throw-away part. Furthermore the required break-off locations are easy to form in plastic. Transparent plastic is particularly suitable in order to permit the visual control on introducing the guide sleeve and the individual instruments. Suitable plastics are polymers such as e.g. PE, PTFE, PP, FEP etc.

It is furthermore preferred for the dilatation pin to comprise at least one cutter for separating open the guide sleeve. Such a cutter usefully extends in the longitudinal direction of the dilatation pin and cuts the guide sleeve on introduction into this. However embodiment forms are also possible in which the guide sleeve has no break-off location and is separated open in its longitudinal direction alone by the cutter provided on the dilatation pin.

The invention further relates to a guide sleeve for a dilatation system as has been described previously. As described, such a guide sleeve according to the invention comprises a break-off location extending in the longitudinal direction of the guide sleeve preferably over its whole length. This permits a tearing-open or widening of the guide sleeve if a dilatation pin is introduced into the guide sleeve. The design of the guide sleeve at the same time corresponds to the previously described construction and preferred embodiment examples of the guide sleeve.

### BRIEF DESCRIPTION OF THE DRAWINGS

The invention is hereinafter described by way of example and by way of the attached figures. In these there are shown in:

FIG. 1 a lateral view of the guide sleeve with an inserted trocar sleeve and veress cannula,

FIG. 2 an enlarged view of the distal end of the guide sleeve in the pierced [inserted] condition,

FIG. 3 schematically, the insertion of a dilatation pin into the guide sleeve,

FIG. 4 the dilatation pin or pushing into the guide sleeve,

FIG. 5 the tearing-open of the guide sleeve on inserting the dilatation pin,

FIG. 6 the guide sleeve in the separated-open condition, with an inserted, larger trocar sleeve,

FIG. 7 the removal of the separated-open guide sleeve,

FIG. 8a and FIG. 8b a first embodiment form of the guide sleeve in a sectional view and a lateral view,

FIG. 9a and FIG. 9b a second embodiment form of the guide sleeve in a sectional view and a lateral view,

FIG. 10 a sectional view of a third embodiment form of the guide sleeve,

FIG. 11 a sectional view of the guide sleeve according to FIG. 10 in a widened condition and

FIG. 12 a lateral view of a dilatation pin with a cutter.

### DETAILED DESCRIPTION OF THE INVENTION

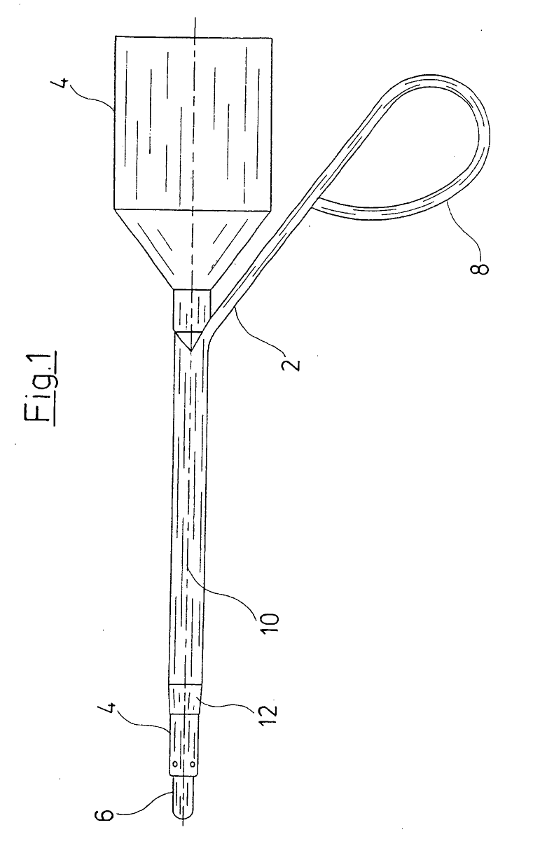

FIG. 1 shows a lateral view of a guide sleeve 2 into which a trocar sleeve 4 is inserted. The trocar sleeve 4 in its inside comprises an optical veress cannula 6 exiting at the distal end. The guide sleeve at its proximal end comprises a holding ring 8 which serves for the improved handling of the guide sleeve 2. Furthermore the guide sleeve 2 in its walling comprises a linear break-off location 10 in the form of a perforation which extends in the longitudinal direction of the guide sleeve. The inner diameter of the guide sleeve 2 corresponds essentially to the outer diameter of the trocar sleeve 4 so that the guide sleeve 2 may be pushed onto the trocar sleeve 4 for introduction into the tissue. At the distal end the guide sleeve 2 comprises a conical chamfering 12 which is to simplify the introduction into the tissue.

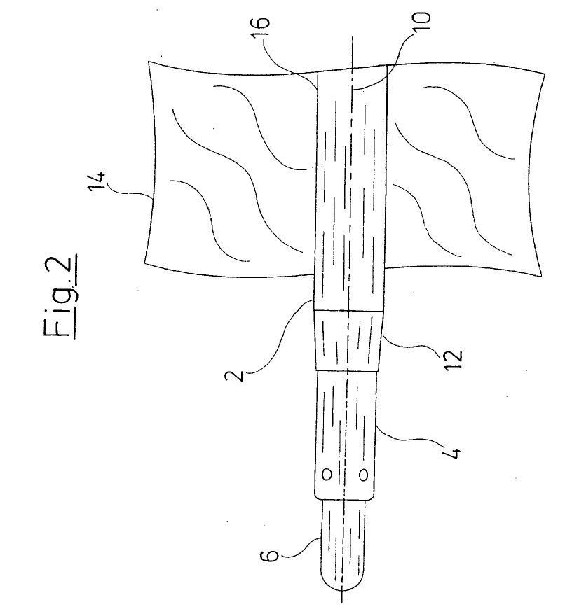

The introduction of the trocar sleeve with the pushed-on guide sleeve 2 into the tissue is explained in more detail by way of FIG. 2. FIG. 2 schematically shows a section through the abdominal wall 14 into which the trocar sleeve 4 with the pushed-on guide sleeve 2 is pierced. On piercing, an optical control is made possible due to the optical veress cannula 6. On piercing the trocar sleeve 4 into the tissue 14 the guide sleeve 2 is simultaneously also introduced into the tissue 14 so that the guide sleeve 2 comes to lie in the created opening 16 in the tissue.

After piercing and introduction of the guide sleeve 2 into the tissue 14 with the help of the trocar sleeve 4, the trocar sleeve 4 and the veress cannula 6 together are proximally withdrawn from the guide sleeve 2 so that only the guide sleeve 2 remains in the opening 16 in the tissue 14 and maintains the opening 16.

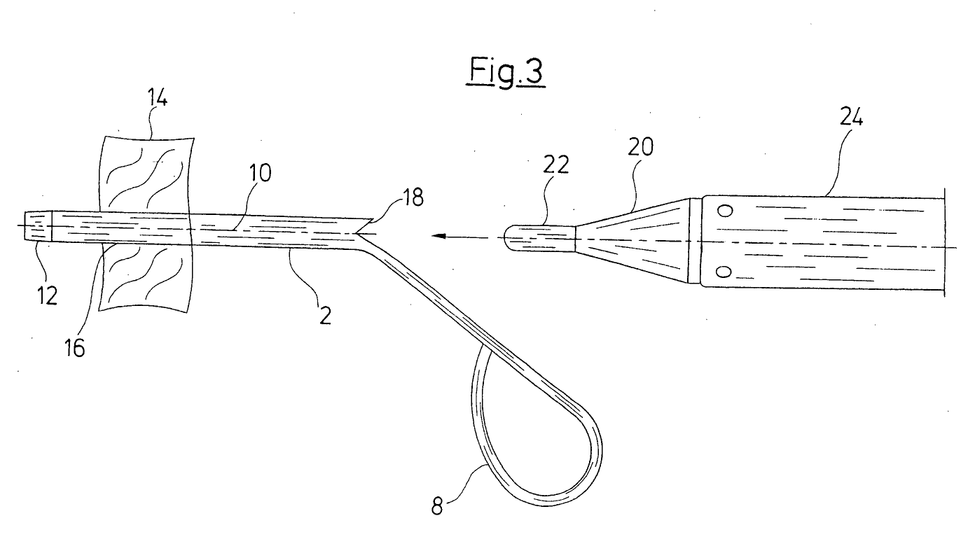

FIG. 3 shows the condition in which the trocar sleeve 4 and the veress cannula 6 have already been withdrawn from the guide sleeve 2 in the proximal direction. If the created opening 16 is now to be widened in order to example to be able to introduce a larger trocar sleeve for an endoscopic operation, a dilatation pin 20 is introduced into the proximal opening 18 of the guide sleeve 2. The dilatation pin 20 is formed conically so that its diameter widens from the distal end in the proximal direction. At the distal end the dilatation pin 20 comprises a guide lug with a small diameter. At the same time the diameter of the guide lug 22 is matched to the inner diameter of the guide sleeve 2 so that the guide lug 22 may be easily introduced into the guide sleeve 2 or may be guided in this. Additionally the guide lug 22 at its distal end is formed atraumatically in order by way of this to be able to follow the course of the guide sleeve without any hindrance. The dilatation pin 20 is arranged within a trocar sleeve 24 with a larger diameter. The trocar sleeve 24 has a larger diameter than the trocar sleeve 4, which renders necessary a widening of the tissue 14 in order to be able to introduce the trocar sleeve 24 into a body cavity. As is indicated in FIG. 3 by the arrow, the dilatation pin 20 with the attached trocar sleeve 24 is introduced in the distal direction into the guide sleeve 2 from its proximal end.

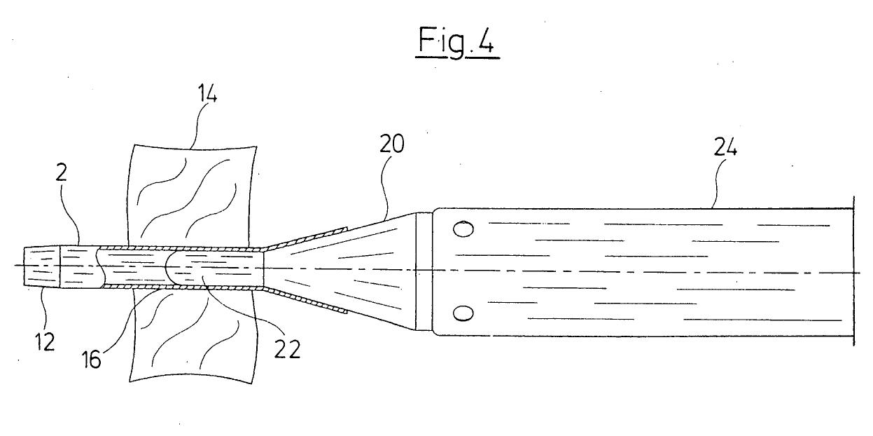

FIG. 4 shows the condition in which the guide lug 22 of the dilatation pin 20 enters into the guide sleeve 2. The guide lug 22 serves for introducing the dilatation pin 20 into the guide sleeve 2 and for the secure guiding through the tissue. The conical, proximally widening section of the dilatation pin 20 enters the guide sleeve 2 at the connection to the guide lug 22 and widens this guide sleeve.

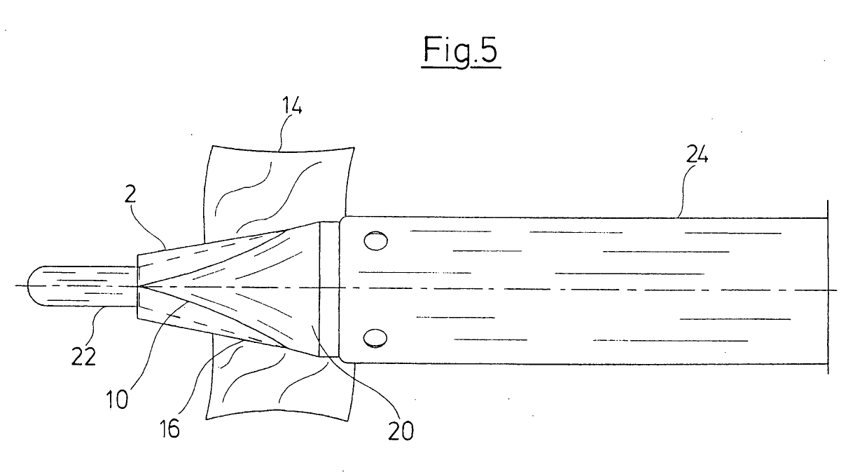

With the further insertion of the dilatation pin 20 in the distal direction, as shown in FIG. 5, the opening 16 in the tissue 14 is likewise widened. At the same time the guide sleeve 2, as shown in FIG. 5, tears along its break-off location or break-off locations 10 linearly over the whole length. This permits the guide sleeve 2 to be accordingly widened or folded out so that the entry of the part of the dilatation pin 20 with a larger diameter and subsequently of the trocar sleeve 24 into the opening 16 it made possible. The guide sleeve 2 on inserting the dilatation pin 20 is firmly held on the holding ring 8 in order to prevent a displacement into the body cavity through the dilatation pin 20.

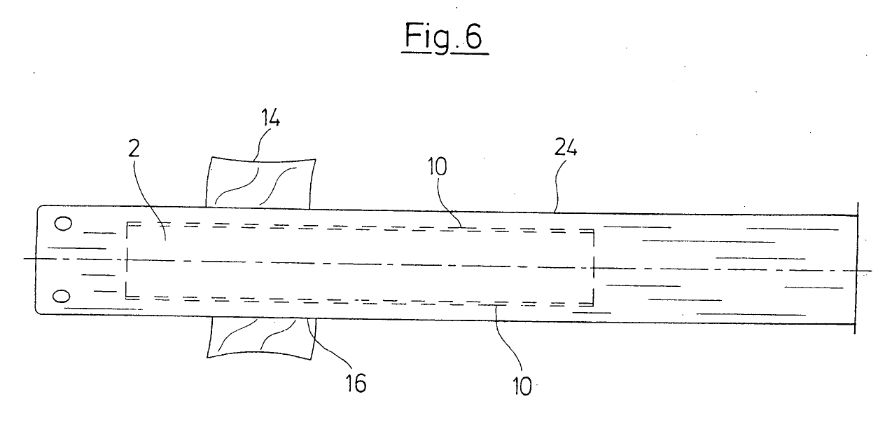

FIG. 6 shows the condition in which the trocar sleeve 24 is completely introduced through the tissue 14 and the dilatation pin 20 is withdrawn from the trocar sleeve 24. The dilatation pin 20 is withdrawn from the trocar sleeve in the proximal direction so that the trocar sleeve 24 at the same time remains in the created opening 16 in the tissue 14. Then further instruments may be introduced through the trocar sleeve 24 into the body cavity for carrying out an endoscopic operation. In the condition shown in FIG. 6 the guide sleeve 2 is torn open over its complete length and now bears on a circumferential section of the trocar sleeve 24 between the trocar sleeve 24 and the tissue 14.

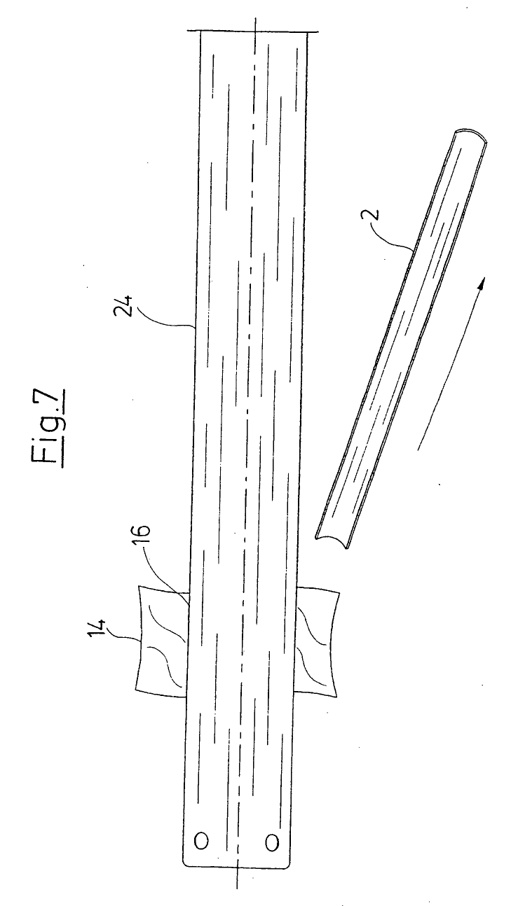

In a next step, which is shown in FIG. 7, the torn-open guide sleeve 2 is withdrawn proximally between the trocar sleeve 24 and the tissue 18, wherein it is gripped on the holding ring 8 which is not shown in FIG. 7. The trocar sleeve 24 with this remains in the created opening 16 in the tissue 14.

The previously described method which is made possible by the dilatation system according to the invention, in particular the guide sleeve 2, ensures that the opening 16 created in the tissue is maintained in order, after the effected removal of the veress cannula 6 and the trocar sleeve 4 with a smaller diameter from the tissue, to be able to carry out the dilatation for introducing the larger trocar sleeve 24.

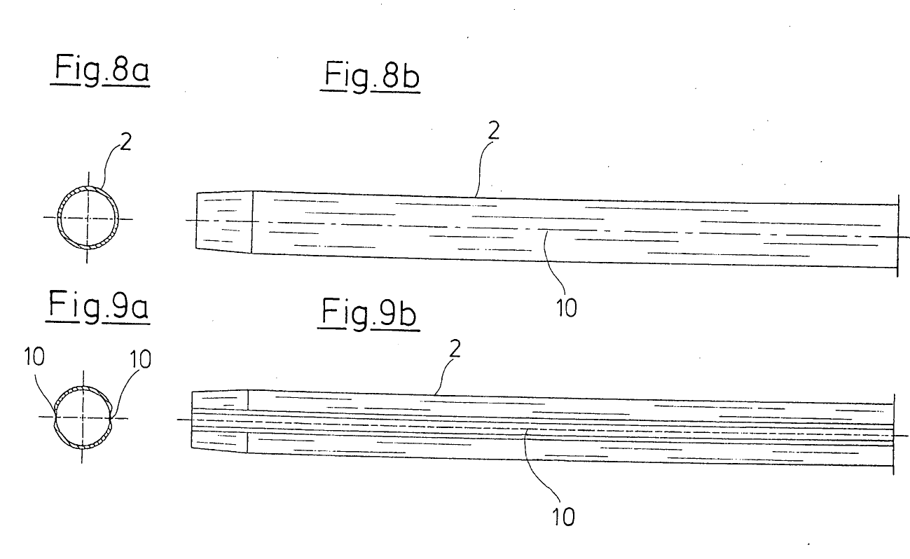

FIG. 8a and FIG. 8b show a first embodiment example of the guide sleeve 2 according to the invention, wherein FIG. 8a shows a sectional view and FIG. 8b a lateral view. The guide sleeve according to FIG. 8a and FIG. 8b over the circumference has a constant material thickness. On one side there is formed a break-off location 10 in the form of a perforation extending in the longitudinal direction of the guide sleeve.

FIG. 9a and FIG. 9b show a second alternative embodiment form of the guide sleeve 2 according to the invention, wherein FIG. 9a shows a sectional view and FIG. 9b a lateral view. With this embodiment from the break-off location 10 extending in the longitudinal direction of the guide sleeve 2 is designed in the form of a weakening of the wall thickness. This means the guide sleeve 2 in the region of the break-off location 10 has a smaller wall thickness than in the remaining circumferential regions. Furthermore this embodiment form comprises two break-of locations 10 arranged diametrically on the circumference of the guide sleeve 2. This has the effect that the guide sleeve 2 falls into two parts on widening by the dilatation pin 20. Preferably accordingly in each case a holding ring 8 may be provided on the two parts in order to be able to easily withdraw the two separated parts of the guide sleeve 2 from the opening 16. Furthermore a combination of the embodiment forms according to FIG. 8a and FIG. 8b with the embodiment forms according to FIG. 9a and FIG. 9b is possible. This means one may create a break-off location 10 which on the one hand has a thinner wall thickness and on the other hand simultaneously has a perforation.

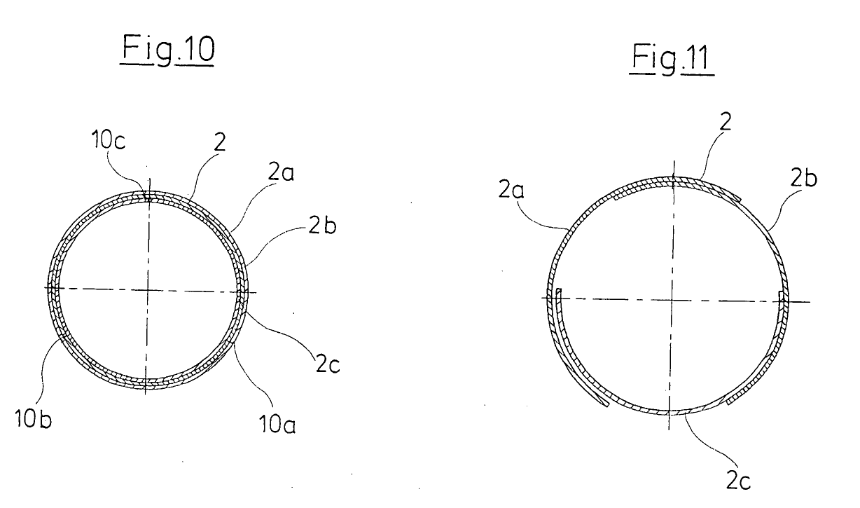

A third embodiment form of the guide sleeve 2 according to the invention is shown by way of FIGS. 10 and 11. FIGS. 10 and 11 show sectional views of this guide sleeve 2, wherein the guide sleeve in FIG. 10 is shown in the closed condition and in FIG. 11 in the widened condition. The guide sleeve according to claim 10 consists of three sleeves 2a, 2b, 2c arranged lying in one another. With this, sleeve 2c has an outer diameter which corresponds essentially to the inner diameter of the sleeve 2b, and the sleeve 2b has an outer diameter which corresponds essentially to the inner diameter of sleeve 2a. This allows the sleeves 2a, 2b and 2c to be arranged lying in one another. At the same time the sleeves 2a, 2b, 2c form separate parts so that they are mutually movable or displaceable in the circumferential direction. Each of the three sleeves 2a, 2b, 2c comprises in each case one break-off location or a slot 10a, 10b and 10c respectively. With this the break-off locations are displaced uniformly over the circumference of the guide sleeve 2 by in each case 120° to one another. This has the effect that each break-off location 10a, 10b and 10c in each case is covered by at least one walling of the respective other sleeve.

FIG. 11 shows the guide sleeve 2 according to FIG. 10 in the widened condition, i.e. after introducing the dilatation pin 20. The sleeves 2a, 2b and 2c widen, wherein at the locations of the break-off locations or slots 10a, 10b, and 10c in each of the sleeves 2a, 2b, and 2c there arise circumferential gaps which however in each case are covered by the walling of one of the other sleeves 2a, 2b and 2c so that a circumferentially closed guide sleeve 2 is maintained even in the widened condition. There are therefore no continuous openings or gaps in the guide sleeve 2 through which tissue may penetrate into the inside of the guide sleeve 2.

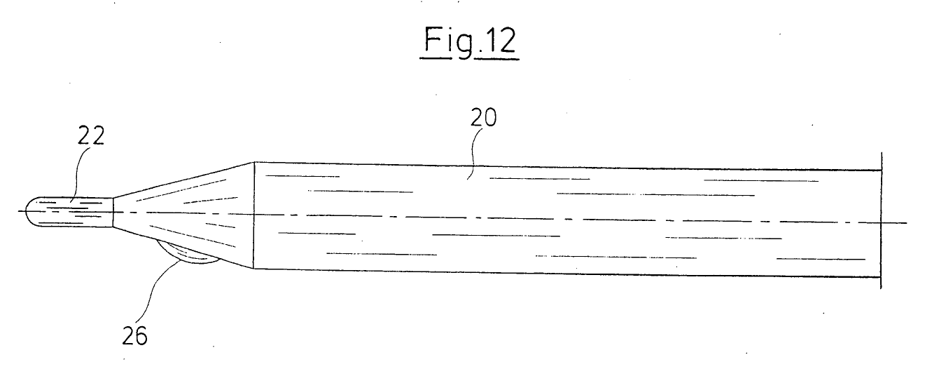

FIG. 12 shows a lateral view of a preferred embodiment form of a dilatation pin 20. The dilatation pin 20 in the region of its cone-shaped or conical section comprises a cutter 26 which extends in the longitudinal direction of the dilatation pin 20 and serves for separating open or cutting open the guide sleeve 2 on inserting into the dilatation pin 20. With this the guide sleeve 2 may be separated open at any location of the circumference even if no corresponding break-off location is provided. Preferably however a break-off location 10 in the guide sleeve 2 is cut open with the cutter 26.

### LIST OF REFERENCE NUMERALS

2—guide sleeve

2a, 2b, 2c—sleeves

4—trocar sleeve

6—veress sleeve

8—holding ring

10—break-off location

10a, 10b, 10c—break-off locations or slots

12—chamfering

14 tissue

16—opening

18—proximal-side opening

20—dilatation pin

22—guide lug

24—trocar sleeve

26—cutter

1. A dilatation system consisting of a dilatation pin (20) with a diameter which increases proximally from the distal end, and with a tubular guide sleeve (2) which has an inner diameter corresponding to the diameter of the distal end (22), reduced in diameter, of the dilatation pin (20), and which in its longitudinal direction may be separated open over the entire length along at least one line (10).

2. A dilatation system according to claim 1, and which the guide sleeve (2) comprises at least one break-off location (10) extending in the longitudinal direction of the guide sleeve (2) preferably over its whole length.

3. A dilatation system according to claim 1 or 2 with a veress cannula (6) which has an outer diameter which corresponds to the inner diameter of the guide sleeve (2).

4. A dilatation system according to one of the preceding claims, with which in the guide sleeve (2) there are formed two preferably diametrically oppositely arranged break-off locations (10) which extend in the longitudinal direction of the guide sleeve (2).

5. A dilatation system according to one of the claims 2 to 4, with which the break-off locations (10) are formed by perforation or a regionally weaker wall thickness of the guide sleeve (2).

6. A dilatation system according to one of the preceding claims, with which the guide sleeve (2) is formed by at least two sleeves (2a, 2b, 2c) which are separate from one another, are arranged in one another and in each case comprise at least one slot extending in the longitudinal direction of the guide sleeve (2) preferably over its whole length or a correspondingly extending break-off location (10a, 10b, 10c), wherein the slots or break-off locations (10a, 10b, 10c) in the two sleeves (2a, 2b, 2c) are arranged circumferentially displaced to one another.

7. A dilatation system according to one of the preceding claims, with which at least one holding element (8) is formed at the proximal end of the guide sleeve (2).

8. A dilatation system according to one of the preceding claims, with which the guide sleeve (2) at its distal end is formed tapered or conically.

9. A dilatation system according to one of the preceding claims, with which the guide sleeve (2) is manufactured of a preferably transparent plastic.

10. A dilatation system according to one of the preceding claims, with which the dilatation pin (20) comprises at least one cutter (26) for separating open the guide sleeve (2).

11. A guide sleeve for a dilatation system according to one of the preceding claims, which comprises at least one break-off location (10) extending in the longitudinal direction of the guide sleeve (2) preferably over its whole length.
